(19) United States
(12) Patent Application Publication
(10) Pub. No.: US 2012/0272981 A1
(43) Pub. Date: Nov. 1, 2012

(54) COSMETIC KIT FOR MAKING UP AND/OR FOR CARE OF KERATINOUS MATERIAL

(75) Inventor: Audrey Ricard, La Varenne (FR)

(73) Assignee: L'OREAL, Paris (FR)

(21) Appl. No.: 13/393,683

(22) PCT Filed: Sep. 10, 2010
(86) PCT No.: PCT/IB2010/054091

§ 371 Date: Jul. 16, 2012

Related U.S. Application Data
(60) Provisional application No. 61/272,379, filed on Sep. 17, 2009.

(30) Foreign Application Priority Data
Sep. 11, 2009 (FR) 0956256

(51) Int. Cl. A45D 34/04; A61Q 1/02; A61K 8/18

(52) U.S. Cl. 132/200; 132/317

(57) ABSTRACT

The present invention provides a cosmetic kit for makeup and/or for care of keratinous material, comprising at least: a container containing at least one cosmetic composition comprising, in a physiologically acceptable medium, at least 16% by weight, preferably at least 18% by weight and more preferably at least 20% by weight of pigments and/or of film-forming polymers and/or of non-pulverulent thickening agents, relative to the total weight of said composition, said composition comprising at least one pigment; an applicator device (8) for applying the composition, provided with an applicator member (10) including an application surface that is capable of turning about at least one axis (x) or center of rotation in response to being moved in engagement with the keratinous material.

The present invention seeks to provide a novel mode for making up and/or for care of keratinous material and more particularly dedicated to applying a composition to a keratinous material, in particular to the skin or the lips, and more particularly to the skin.

In general, cosmetic compositions must bestow an esthetic effect on application and maintain that esthetic effect over time. They must in particular resist the various external factors that could modify their esthetic effect, such as perspiration or tears for a foundation, or saliva for a lipstick.

In particular, makeup compositions, in particular of the foundation type, are expected to produce a certain degree of coverage. Such compositions may advantageously be used with the aim of masking color imperfections of the face and body such as rings under the eyes, blotches, age spots and birthmarks, or spots linked to a disorder such as vitiligo.

Furthermore, once applied to the skin, such compositions must have good wear properties; in particular color, mattness, and uniformity must be long-lasting.

Various formulations for making up and/or for care of keratinous material, in particular foundations, are known to favor wear and/or coverage properties.

However, for the reasons mentioned below, such compositions generally cannot satisfy all of the requirements mentioned above.

Thus, a large number of compositions, such as foundations, include film-forming polymers and/or specific thickening agents specifically intended to increase their wear. Such polymers and/or thickening agents may have a variety of chemical natures and are generally carried either in a fatty phase or in an aqueous phase.

However, while such polymers and/or thickening agents effectively provide the compositions with improved wear properties, they may suffer from problems in terms of application comfort. Specifically, on application of the composition, they may generate a tacky texture that renders smoothing of the composition difficult. Moreover, that tacky sensation, felt by the user during application of the composition with the palm or finger generally serving as applicator means, renders the composition unattractive.

In general, foundation compositions are also rich in volatile oils that evaporate off rapidly following application of the composition, resulting in reduction of the liquid fraction and in concentration of the deposit.

Such compositions, which dry very rapidly, may prove to be difficult to apply uniformly. The user has a limited time available for making the thickness of the deposit uniform once the composition has been applied.

Finally, improvements in the coverage properties of compositions generally involve increasing the quantity of pigments such as oxides of iron or titanium in the makeup compositions.

However, such compositions are generally thick and difficult to apply because of the high pigment content, which may result in an application procedure and/or a makeup that is not always uniform. The high viscosity of such compositions also makes it difficult to use conventional applicator means such as sponges or brushes.

For obvious reasons, it would be desirable to be able to benefit from the specific advantages of the above-mentioned compositions, namely in terms of coverage properties and/or wear properties without, however, being affected by their disadvantages in terms of application difficulties and the quality of the final makeup, especially the uniformity of the deposit.

The present invention is precisely aimed at responding to this need.

More particularly, exemplary embodiments of the present invention provide a cosmetic kit for making up and/or for care of keratinous material, the assembly comprising at least:
a container containing at least one cosmetic composition comprising, in a physiologically acceptable medium, at least 16% by weight, preferably at least 18% by weight and more preferably at least 20% by weight of pigments and/or of film-forming polymers and/or of non-pulverulent thickening agents, relative to the total weight of said composition; and
an applicator device for applying the composition, provided with an applicator member including an application surface that is capable of turning about at least one axis or center of rotation in response to being moved in engagement with the keratinous material.

In particular, the composition according to the invention comprises at least one pigment.

More particularly, a composition of the invention is fluid.

In the meaning of the present invention, the term “fluid” is used to mean a composition that is capable of flowing under its own weight, as opposed to compositions that are termed solid.

The fluid composition of the invention may be viscous to a greater or lesser extent, of a creamy or pasty appearance. It may also be in the form of a gel.

The composition of the invention may be a composition for making up and/or for care of the skin, in particular the face and/or the body, and may constitute a blusher, an eye shadow, a foundation, a concealer, a lipstick, a body makeup composition, a face or body care product or a sunscreen.

More particularly, but not exclusively, the invention provides a foundation composition.

The applicator device is not limited to a single variant embodiment of the applicator member.

As can be seen from the accompanying figures, several variant embodiments may be envisaged provided that they are compatible with rotation of their application surface about at least one axis or center of rotation in response to said surface moving in contact with the keratinous material to be treated, generally the skin.

The applicator member may be defined by a roller, a ball, or a band disposed about two rollers with parallel axes of rotation.

The container containing the cosmetic composition, in particular in the form of a fluid foundation, may optionally be integral with the applicator member during use or storage.

The applicator member is intended both to take a portion of the composition present in the associated container and to apply the composition taken thereby to the surface of a keratinous material.

Firstly, the outer surface of the applicator member, and more particular of the roller, is impregnated or coated with composition. The applicator member may be used to take the composition from a distinct container, or the composition may be delivered directly to the application surface by means of a container containing the composition to be applied and that is integral with said applicator member.

Secondly, the consecutive passage of the outer surface of the applicator member, more particularly of the roller, on the surface of a keratinous material, in particular the skin of the face, causes it to rotate and smoothes the composition disposed on its outer surface.

In variant exemplary embodiments, the applicator member may be provided with a releasable brake that can block or brake free rotation thereof, if necessary, during removal and/or application.

Having regard to the various variant embodiments proposed below, in first variant exemplary embodiments these two steps, i.e. impregnation of the applicator member with the composition and application to the keratinous material, may be carried out consecutively. This happens when the container containing the composition is distinct from the applicator member. The user must initially bring the outer surface of the applicator member into contact with the composition to be taken. Depending on the texture of the composition, in particular its fluidity and viscosity, the applicator member may be impregnated and/or coated with the composition by rubbing and/or pressing the surface of the applicator member onto the surface of the composition, which may possibly cause it to rotate, or alternatively it may be “immersed” in the composition.

In other variant exemplary embodiments, impregnation of the applicator member with the composition and its movement over the keratinous material may be carried out simultaneously, as happens with applicator devices of the “distributor-applicator” tube type shown in FIG. 6, in which the composition to be applied is stored in a container that is integral with the applicator member and it is delivered directly to the surface of the applicator member.

The outer face of the applicator member is formed, at least on the surface, by a material that is capable of removing cosmetic composition and of retaining it until it is subsequently applied to the skin.

For example, the application surface intended to come into contact with a keratinous material may in particular be a foam with optionally-flocked open or closed cells, a flocked material, an elastomer, a sintered material, a woven material, or a nonwoven material.

The outer surface of the applicator member may optionally be smooth. Thus, said applicator member may advantageously have surface portions in relief that are generally domed and rounded, which portions are advantageous in providing a simultaneous massaging effect.

In preferred variant exemplary embodiments, its appearance is close to that of an applicator roller similar to that used for applying paint. Such applicators used for cosmetic purposes are described in particular in documents FR 985 064 (lipstick), FR 1 524 192 (powder), FR 1 281 338 (compact), FR 2 848 790 (solid or fluid cosmetic) and WO 2006/090061 (fluid).

Thus, the applicator member is advantageously in the form of a roller that may optionally be hollow and that rotates about an axis of rotation. Said axis of rotation may advantageously be disposed perpendicular to the longitudinal axis of the device.

In particular exemplary embodiments, the applicator device and the container are combined in the same packaging, for example the same case provided with a base containing the composition and having a recess for receiving the applicator and a cover that can move relative to the base, for example being hinged thereto.

In a particularly preferred embodiment, the application surface of said applicator device is of the foam type, thereby different from plastic or metal materials generally used for rotating balls of roll-on dispensing systems, like roll-on deodorants and antiperspirants.

In an other particularly preferred embodiment, the applicator means are advantageously in the form of a roller, thus different from a rotating ball.

In particular, the use of a roller presenting a larger application surface in contact with the keratinous material makes it possible to improve the quality of the final makeup, especially in terms of uniformity of the deposit.

During application, the applicator member is capable of turning on the skin without slipping. Several passes in succession may be made at the same location depending, for example, on the desired intensity of color. During successive passes, the user may modify the direction of rolling a little in order to blend out edges of the deposited composition.

Using an applicator member of the invention facilitates removal and application of the composition, in particular, ease of application is improved, in particular with compositions incorporating large quantities of pigments and/or film-forming polymers and/or non-pulverulent thickening agents, which in particular may have high viscosity.

Such an applicator device is easily held in the hand and very easy to use. It also has the advantage of not soiling the fingers with foundation composition and it avoids a stage of applying and smoothing viscous and/or tacky-textured compositions with the palm or the fingers, something that is disagreeable to the user.

Further, using an applicator member of the invention enables a makeup effect to be obtained that is significantly improved in terms of uniformity of the deposit.

The present invention thus enables the advantageous wear and/or coverage properties of the film of makeup obtained with compositions that are particularly rich in pigments and/or in film-forming polymers and/or in non-pulverulent thickening agents, to be combined with ease of application and good uniformity of the deposit.

Finally, using an applicator member of the invention to deposit a composition on the surface of a keratinous material, more particularly the skin, may produce a massaging effect on the skin that may produce sensations that are agreeable to the user.

In other exemplary embodiments the invention provides a makeup and/or care method for a keratinous material, the method comprising the steps of:
taking a quantity of a composition, in particular in the form of fluid foundation, with the aid of an applicator member including an application surface that is capable of turning about an axis or a center of rotation in particular as defined below, in response to being moved in engagement with the keratinous material; and
applying the composition as taken in this way to said keratinous material with the aid of said applicator member, said composition including at least 16% by weight, preferably at least 18% by weight and more preferably at least 20% by weight of pigments and/or of film-forming polymers and/or of non-pulverulent thickening agents, relative to the total weight of said composition.

### EXAMPLES OF APPLICATOR DEVICES AND OF COSMETIC KITS FOR CARRYING OUT THE INVENTION

The figures show some examples, amongst others, of applicator devices that may be used of the invention, and that are particularly adapted to the applying of a fluid composition with greater or lesser viscosity.

In the drawings:

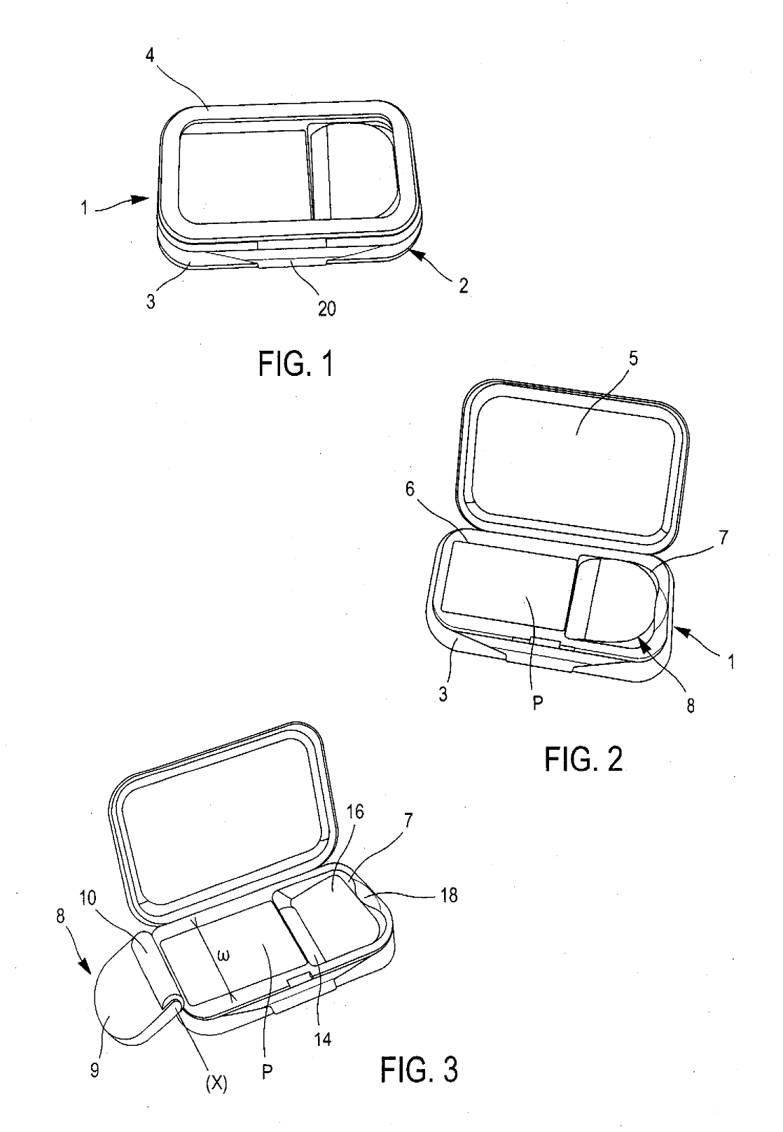

FIG. 1 is a diagrammatic perspective view of an example of a packaging and applicator device of the invention;

FIG. 2 shows the FIG. 1 device, with its case open;

FIG. 3 shows the FIG. 1 device, with its applicator device removed;

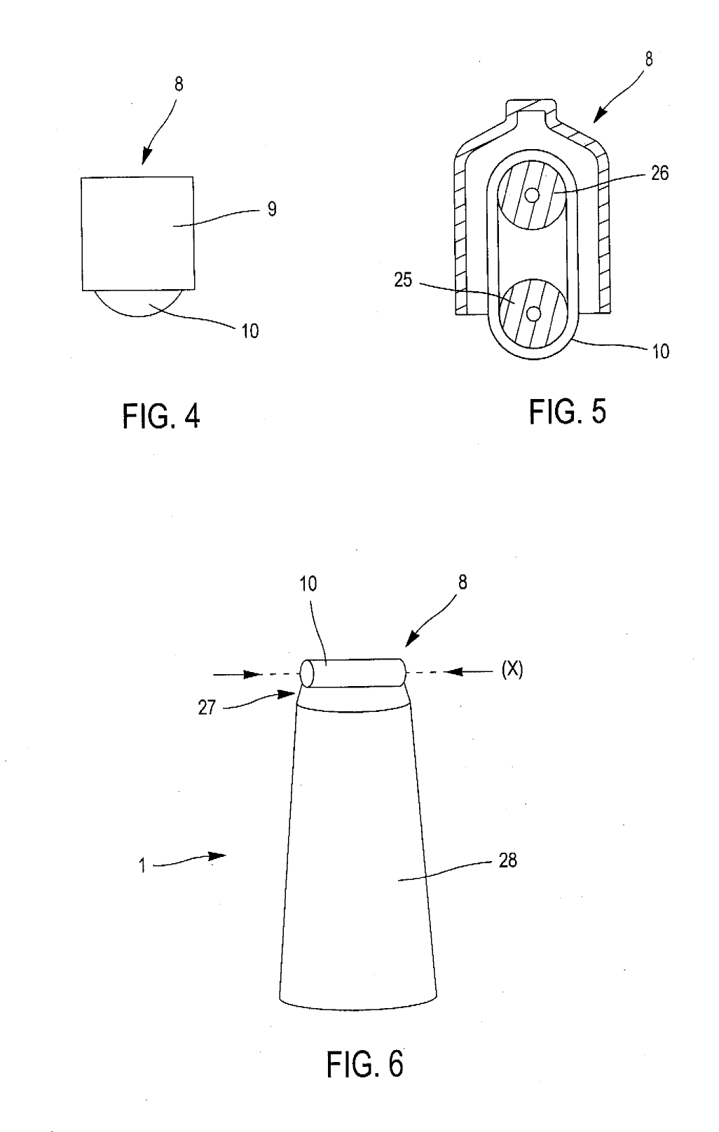

FIG. 4 is a diagrammatic elevation view of a variant embodiment of the applicator member;

FIG. 5 shows another variant embodiment of the applicator device, in diagrammatic, fragmentary longitudinal section;

FIG. 6 is a diagrammatic perspective view of another example of a packaging and applicator device of the invention; and

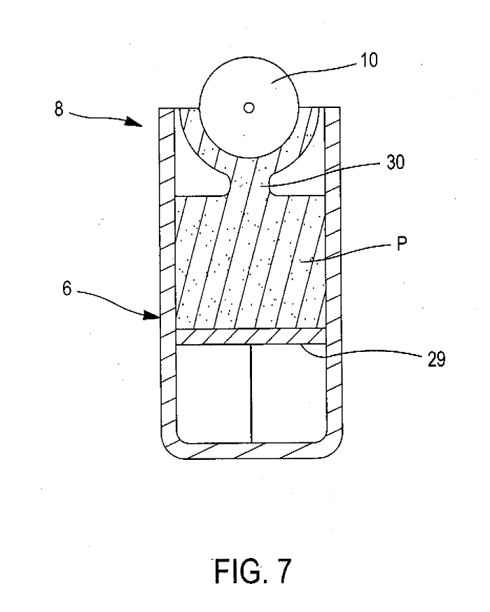

FIG. 7 shows another variant embodiment of the applicator device, in diagrammatic, fragmentary longitudinal section.

The packaging and applicator device 1 shown in FIGS. 1, 2, and/or 3 comprises a case 2 comprising a base and a lid 4 that is movable relative to the base; said lid 4 may comprise a transparent window 5 allowing the contents of the case to be seen when the case is closed.

The base 3 includes a recess 6 receiving the composition of the invention and a recess 7 accommodating an applicator device 8, acting to take the composition P and to apply it to human keratinous material.

The applicator device 8 comprises a handle 9 and an applicator member 10 capable of turning relative to the handle 9, for example and as shown, about an axis of rotation X that may be oriented perpendicularly to the longitudinal axis of the handle. This handle may have a shape that is generally flat parallel to a plane containing the axis of rotation X.

The applicator member 10 may be in the form of a roller with an application surface that may be a cylinder of revolution about the axis X.

The application surface may be defined by any material capable of retaining the composition and of applying it, for example an elastomeric material or a foam with open or closed cells, or a flocked membrane. The material defining the application surface may be compressible.

The length of the applicator member 10 may correspond substantially to the width w of the recess 6 containing the composition.

The recess 7 receiving the applicator device 8 may have a bottom 14 provided with a grid 14 and may include a slope 16 that keeps the handle 9 inclined slightly upwardly and towards the side of the case, to facilitate grasping by the user. A cutout 18 may open into the recess 7 in order to facilitate engagement of a finger in order to pick up the handle 9.

The recess 6 receiving the composition P may be parallelepipedal in shape or may have a concave rounded shape that is a portion of a cylinder with a generator line parallel to the axis of rotation X when the applicator device 8 is moved in the recess 6 containing the composition in order to load it with composition. This should avoid the presence of corners where the composition could accumulate without being taken.

The case may include a clasp 20 that may be of any type.

To use the applicator device 8, the user brings the applicator member 10 into contact with the composition P contained in the container 6, by moving it to a greater or lesser depth in the container 6, or over the surface of the composition, P, and then applying it to the keratinous material to be treated, for example the skin, by passing it one or more times over the zone where the composition is to be deposited.

During movement over the skin, the applicator member 10 can roll on without slipping.

The invention is not limited to an applicator member in the form of a roller turning about an axis of rotation.

As an example, as shown in FIG. 4, the applicator member 10 may be in the form of an applicator ball 10, which may be retained in a cage, not shown, that is fastened to a handle 9. In the example of FIG. 4, the applicator member 10 is capable of turning about any axis in its cage.

In a variant embodiment shown in FIG. 5, the applicator member 10 can also be defined by an applicator band mounted to turn about two rollers 25 and 26 with parallel axes of rotation. The portion of the band 10 acting in application may be moved in contact with the skin when the applicator device 10 is moved in contact therewith to apply the composition.

In a variant embodiment, not shown, the axes of rotation of the rollers 25 and 26 are disposed in such a manner that the portion of the band 10 that comes into contact with keratinous material in order to apply the composition is not in contact with only one roller but is between both rollers.

In a variant embodiment, the packaging and applicator device 1 shown in FIG. 6 is in the form of a distributor-applicator tube adapted for storage, distributing and applying a fluid composition P of greater or lesser viscosity.

Such applicator-distributor tubes are described in particular in patent application WO 2006/090061.

Such a device may comprise a composition-storing container in the form of a flexible tube 28 including at its end a tip 27 having an applicator device 8.

The flexible tube 28 may take any form and may be made of various different materials, especially plastics or thermoplastic materials.

Advantageously, the tip is surmounted by a suitable cap, not shown, that protects the applicator device during storage of the tube.

The applicator device 8 of the invention includes an applicator member 10 that may be in the form of a roller having an application surface that may be a cylinder of revolution about the axis X, as shown in FIG. 6. This applicator member is capable of turning and can take the composition from the container on one side while applying it on its other side.

In a variant embodiment, not shown, the applicator member of the tip of the distribution-application tube may be an applicator ball as can be seen in FIG. 4.

In one possible variant embodiment of the packaging and applicator device shown in FIG. 6, the tip 27 may include a hollow compartment, not shown, connected with the interior of said tube 28. The applicator roller 10 can thus turn in said hollow compartment, supplied with composition, and transport the composition to the keratinous material.

To use the packaging and applicator device shown in FIG. 6, and in order to load the tip 27 with composition, the user may press on the flexible wall of the tube 28 while continuing to apply said applicator member 8 to the keratinous material to be treated, for example the skin.

In yet another variant embodiment, the packaging and application kit shown in FIG. 7 may include a container 6 for storing the composition P, and in which a piston 29 may slide to force the composition P towards the applicator member 10 that are supplied with the composition via at least one internal channel 30. The applicator member 10 may be as defined above, for example a ball.

In variant embodiments that are not shown, the applicator device 8 may be provided with a brake to brake or even prevent the rotation of the applicator member 10 relative to the handle.

Viscosity

A composition of the invention may include at least one pigment and/or at least one film-forming polymer and/or at least one non-pulverulent thickening agent in quantities as defined below.

The compositions of the invention, when they have a high pigment content, are generally thick and have high viscosity.

More particularly, a composition of the invention including at least 16% by weight, preferably at least 18% by weight, and more preferably at least 20% by weight, of pigments relative to the total weight of said composition, and at 25° C. and at a shear rate=1 s⁻¹ [reciprocal second], may have viscosity of more than 3 Pa·s [pascal-second], in particular lying in the range 3 Pa·s to 6 Pa·s. It is preferably measured using a RHEOMAT CONTRAVE viscosimeter provided with a no. 4 moving body. Such a composition is described in Example 1 below.

When compositions of the invention contain pigments and a large quantity of film-forming polymers and/or thickening agents, they may have lower viscosities but, in contrast, they suffer from the disadvantage of drying very rapidly on application and/or of having a tacky nature. Such compositions are described in Examples 2 and 3 below.

Pigments

In variant exemplary embodiments, a composition of the invention may include at least one pigment.

The term “pigments” should be understood to mean particles of any form, whether white or colored, or mineral or organic, that are insoluble in the physiological medium, and that are intended to color the composition.

The pigments may be white or colored, mineral and/or organic.

Mineral pigments that may be mentioned include titanium dioxide, optionally surface treated, oxides of zirconium or cerium, as well as oxides of zinc, iron (black, yellow, or red) or of chromium, manganese violet, ultramarine blue, chromium hydrate and ferric blue, metal powders such as aluminum powder, and copper powder.

The pigments may also have a structure that may, for example, be of the sericite, brown iron oxide, titanium dioxide, silica type. Such a pigment is, for example, sold under the reference COVERLEAF NS or JS by the supplier CHEMICALS AND CATALYSTS.

It is also possible to mention a pigment with a structure that may, for example, be of the silica microsphere type containing iron oxide. An example of a pigment having this structure is that from the supplier MIYOSHI under the reference PC BALL PC-LL-100 P, this pigment being constituted by silica microspheres containing yellow iron oxide.

Organic pigments that may be used in the invention and that may be mentioned are carbon black, D & C type pigments, lakes based on carmine and cochineal, barium, strontium, calcium, aluminum or diketopyrrolopyrrole (DPP) described in the documents EP-A-0 542 669, EP-A-0 787 730, EP-A-0 787 731 and WO-A-96/08537.

In particularly preferred exemplary embodiments, a composition of the invention includes at least one pigment selected from yellow, red or black oxides of iron and titanium dioxide, in particular coated with at least one hydrophobic agent, especially an aluminum acyl glutamate, and more particularly aluminum stearoyl glutamate, in particular those sold under the reference NAI-C33-7001-10 by the supplier Myoshi Kasei.

The pigments may be present in a composition of the invention in a quantity lying in the range 8% to 40% by weight, in particular in the range 10% to 30% by weight, and in particular in the range 10% to 25% by weight, relative to the total weight of said composition, provided that the total quantity of pigment(s) and/or film-forming polymer(s) and/or thickening agent(s) is greater than or equal to 16% by weight, preferably greater than or equal to 18% by weight, and more preferably greater than or equal to 20% by weight, relative to the total weight of the composition.

In particular, the quantity of pigments may be greater than or equal to 16% by weight, preferably greater than or equal to 18% by weight, and more preferably greater than or equal to 20% by weight, relative to the total weight of said composition.

In a particular embodiment, a composition of the invention may include a total quantity of pigments greater than or equal to 20% by weight relative to the total weight of the composition, preferably in the range 22% to 65% by weight, and more preferably in the range 24% to 45% by weight.

Additional Pulverulent Colorants

The pulverulent phase of a composition of the invention may further comprise additional colorants such as nacres, pigments with an optical effect, and mixtures thereof.

The term “nacres” means colored particles of any form, iridescent or otherwise, produced in the shells of certain mollusks or synthetically; their color effect is produced by optical interference.

The nacres may be selected from nacrous pigments such as mica titanium covered with chromium oxide, mica covered with bismuth oxychloride, mica titanium covered with chromium oxide, mica titanium covered with an organic colorant and nacrous pigments based on bismuth oxychloride. The nacres may also be mica particles having at least two successive layers of metallic oxides and/or of organic colorants superposed on the surface thereof.

Examples of nacres that may be mentioned are natural mica covered with titanium oxide, iron oxide, natural pigment or bismuth oxychloride.

Commercially available nacres that may be mentioned are TIMICA, FLAMENCO and DUOCHROME (mica-based) from the supplier ENGELHARD, TIMIRON nacres from the supplier MERCK, PRESTIGE mica-based nacres from the supplier ECKART and SUNSHINE synthetic mica-based nacres from the supplier SUN CHEMICAL.

More particularly, the nacres may have a yellow, pink, red, bronze, orangey, brown, gold and/or coppery glint or color.

The term “material with a specific optical effect” means a material with an effect that differs from a simple hue effect that is conventional, i.e. unitary and stable, such as that produced by conventional colorants such as monochromatic pigments, for example. In the context of the invention, “stabilized” means free of the effect of color varying with the angle of observation or in response to a change in temperature.

As an example, this material may be selected from particles with a metallic glint, goniochromatic coloring agents, diffracting pigments, thermochromic agents, optical whitening agents, as well as fibers, in particular interference fibers.

Said additional pulverulent colorants may represent in the range 0.1% to 40% by weight, especially in the range 1% to 30% by weight, and in particular in the range 5% to 15% by weight, relative to the total weight of the cosmetic composition.

In other exemplary embodiments of the invention, the composition of the invention may comprise a total quantity of film-forming polymers and/or non-pulverulent thickening agents that is greater than or equal to 10% by weight, relative to the total weight of the composition, preferably in the range 10% to 30% by weight, and more preferably in the range 12% to 20% by weight.

Film-Forming Polymers

The term “polymer”, as used herein, means a composition containing at least two repeat motifs, preferably at least three repeat motifs.

The term “film-forming polymer” designates a polymer capable on its own or in the presence of an auxiliary film-forming agent, of forming a macroscopically continuous film on a support, especially on keratinous material.

Film-forming polymers suitable for use in the compositions of the present invention and that may be mentioned are synthetic polymers, of the radical or polycondensate type, polymers of natural origin, and mixtures thereof.

The term “radical film-forming polymer” means a polymer obtained by polymerization of monomers with an unsaturated bond, in particular an ethylene bond, each monomer being capable of being homopolymerized (in contrast to polycondensates).

Radical type film-forming polymers may in particular be vinyl polymers or copolymers, in particular acrylic polymers.

Vinyl film-forming polymers may result from the polymerization of monomers with an ethylene type unsaturated bond containing at least one acid group and/or esters of these acid monomers and/or amides of these acid monomers.

As the monomer carrying an acid group, it is possible to use unsaturated α,β-ethylene type carboxylic acids such as acrylic acid, methacrylic acid, crotonic acid, maleic acid or itaconic acid. Preferably, (meth)acrylic acid and crotonic acid, and more preferably (meth)acrylic acid, are used.

The acid monomer esters are advantageously selected from esters of (meth)acrylic acid (also termed (meth)acrylates), especially alkyl(meth)acrylates, in particular C₁-C₃₀ and preferably C₁-C₂₀ alkyl, aryl(meth)acrylates, in particular C₆-C₁₀ aryl, hydroxyalkyl(meth)acrylates, in particular C₂-C₆ hydroxyalkyl.

Alkyl(meth)acrylates that may be mentioned are methyl methacrylate, ethyl methacrylate, butyl methacrylate, isobutyl methacrylate, 2-ethylhexyl methacrylate, lauryl methacrylate and cyclohexyl methacrylate.

Hydroxyalkyl(meth)acrylates that may be mentioned are hydroxyethyl acrylate, 2-hydroxypropyl acrylate, hydroxyethyl methacrylate and 2-hydroxypropyl methacrylate.

Aryl(meth)acrylates that may be mentioned are benzyl acrylate and phenyl acrylate.

Particularly preferred (meth)acrylic acid esters are alkyl(meth)acrylates.

In the present invention, the alkyl group of the esters may be either fluorinated or perfluorinated, i.e. all or some of the hydrogen atoms of the alkyl group are substituted with fluorine atoms.

Examples of acid monomer amides that may be mentioned are (meth)acrylamides, and in particular N-alkyl(meth)acrylamides, in particular C₂-C₁₂ alkyl. N-alkyl(meth)acrylamides that may be mentioned are N-ethyl acrylamide, N-t-butyl acrylamide, N-t-octyl acrylamide and N-undecylacrylamide.

Vinyl film-forming polymers may also result from homopolymerization or copolymerization of monomers selected from vinyl esters and styrene monomers. In particular, these monomers may be polymerized with acid monomers and/or their esters and/or their amides as mentioned above.

Examples of vinyl esters that may be mentioned are vinyl acetate, vinyl neodecanoate, vinyl pivalate, vinyl benzoate and vinyl t-butyl benzoate.

Styrene monomers that may be mentioned are styrene and alpha-methyl styrene.

Film-forming polycondensates that may be mentioned are polyurethanes, polyesters, polyester amides, polyamides, and epoxyester resins and polyureas.

The polyurethanes may be selected from anionic, cationic, non-ionic or amphoteric polyurethanes, polyurethane-acrylics, polyurethane-polyvinylpyrrolidones, polyester-polyurethanes, polyether-polyurethanes, polyureas, polyurea-polyurethanes, and mixtures thereof.

The polyesters may be obtained, in known manner, by polycondensation of dicarboxylic acids with polyols, in particular diols.

The dicarboxylic acid may be aliphatic, alicyclic or aromatic. Examples of such acids that may be mentioned are: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornane dicarboxylic acid, diglycolic acid, thiodipropionic acid, 2,5-naphthalenedicarboxylic acid and 2,6-naphthalenedicarboxylic acid. These dicarboxylic acid monomers may be used alone or in a combination of at least two dicarboxylic acid monomers. Of these monomers, preferred monomers are phthalic acid, isophthalic acid, and terephthalic acid.

The diol may be selected from aliphatic, alicyclic and aromatic diols. Preferably, a diol is used that is selected from: ethylene glycol, diethylene glycol, triethylene glycol, 1,3-propanediol, cyclohexane dimethanol and 4-butanediol. Other polyols that may be used are glycerol, pentaerythritol, sorbitol and trimethylol propane.

The polyester amides may be obtained in a manner that is analogous to the polyesters, by polycondensation of diacids with diamines or amino alcohols. Diamines that may be used are ethylenediamine, hexamethylenediamine and meta- or para-phenylenediamine. An aminoalcohol that may be used is monoethanolamine.

The polyester may further comprise at least one monomer carrying at least one —SO₃M group, with M representing a hydrogen atom, an ammonium ion NH₄⁺ or a metallic ion, such as a Na⁺, Li⁺, K⁺, Mg²⁺, Ca²⁺, Cu²⁺, Fe²⁺ or Fe³⁺ ion, for example. In particular, a bifunctional aromatic monomer including such a —SO₃M group may be used.

The aromatic ring of the bifunctional aromatic monomer further carrying a —SO₃M group as described above may, for example, be selected from benzene, naphthalene, anthracene, diphenyl, oxydiphenyl, sulfonyldiphenyl and methylenediphenyl nuclei. Examples of this bifunctional aromatic monomer further carrying a —SO₃M group that may be mentioned are: sulfoisophthalic acid, sulfoterephthalic acid, sulfophthalic acid and 4-sulfonaphthalene-2,7-dicarboxylic acid.

Preferably, copolymers based on isophtalate/sulfoisophtalate are used, more particularly copolymers obtained by condensation of diethyleneglycol, cyclohexane dimethanol, isophthalic acid and sulfoisophthalic acid.

The optionally modified polymers of natural origin, may be selected from shellac resin, sandarac gum, dammars, elemis, copals, cellulosic polymers, and mixtures thereof.

In first exemplary embodiments of the invention, the film-forming polymer may be a hydrosoluble polymer and may then be present in the continuous aqueous phase of an emulsion of the invention.

In other variant exemplary embodiments, the film-forming polymer may be a polymer dissolved in an oily liquid phase comprising oils or organic solvents such as those described below (the film-forming polymer is then said to be a liposoluble polymer). Preferably, the liquid fatty phase comprises a volatile oil, optionally mixed with a non-volatile oil; the oils may be selected from the oils mentioned below.

Examples of liposoluble polymers that may be mentioned are vinyl ester copolymers (the vinyl group being directly bonded to the oxygen atom of the ester group and the vinyl ester having a linear or branched saturated hydrocarbon radical containing 1 to 19 carbon atoms, bonded to the carbonyl of the ester group) and at least one other monomer that may be a vinyl ester (different from the already present vinyl ester), an α-olefin (containing 8 to 28 carbon atoms), an alkylvinylether (in which the alkyl group contains 2 to 18 carbon atoms), or an allyl or methallyl ester (containing a saturated, linear or branched hydrocarbon radical containing 1 to 19 carbon atoms, bonded to the carbonyl of the ester group).

These copolymers may be cross-linked with the aid of cross-linking agents that may either be of the vinyl type or of the allyl or methallyl type, such as tetraallyloxyethane, divinylbenzene, divinyl octanedioate, divinyl dodecanedioate, or divinyl octadecanedioate.

Examples of these copolymers that may be mentioned are the following copolymers: vinyl acetate/allyl stearate, vinyl acetate/vinyl laurate, vinyl acetate/vinyl stearate, vinyl acetate/octadecene, vinyl acetate/octadecylvinylether, vinyl propionate/allyl laurate, vinyl propionate/vinyl laurate, vinyl stearate/octadecene-1, vinyl acetate/dodecene-1, vinyl stearate/ethylvinylether, vinyl propionate/cetyl vinyl ether, vinyl stearate/allyl acetate, 2,2-dimethyl vinyl octanoate/vinyl laurate, 2,2-dimethyl allyl pentanoate/vinyl laurate, dimethyl vinyl propionate/vinyl stearate, dimethyl allyl propionate/vinyl stearate, vinyl propionate/vinyl stearate, cross-linked with 0.2% of divinylbenzene, vinyl dimethyl propionate/vinyl laurate, cross-linked with 0.2% of divinylbenzene, vinyl acetate/octadecyl vinyl ether, cross-linked with 0.2% de tetraallyloxyethane, vinyl acetate/allyl stearate, cross-linked with 0.2% of divinylbenzene, vinyl acetate/octadecene-1 cross-linked with 0.2% of divinylbenzene and allyl propionate/allyl stearate cross-linked with 0.2% of divinylbenzene.

Examples of liposoluble film-forming polymers that may also be mentioned are liposoluble copolymers, in particular those resulting from copolymerization of vinyl esters containing 9 to 22 carbon atoms or of acrylates or of alkyl methacrylates, the alkyl radicals containing 10 to 20 carbon atoms.

Such liposoluble copolymers may be selected from copolymers of polyvinyl stearate, polyvinyl stearate cross-linked with the aid of divinylbenzene, diallylether or diallyl phthalate, copolymers of stearyl poly(meth)acrylate, polyvinyl laurate, lauryl poly(meth)acrylate; these poly(meth)acrylates may be cross-linked with the aid of ethylene glycol dimethacrylate or tetraethylene glycol.

The liposoluble copolymers defined above are known and in particular described in application FR-A-2 232 303; they may have a mass average molecular weight in the range 2000 to 500,000, preferably from 4000 to 200,000.

It is also possible to mention liposoluble homopolymers, and in particular those resulting from the homopolymerization of vinyl esters containing 9 to 22 carbon atoms or alkyl acrylates or methacrylates, the alkyl radicals containing 2 to 24 carbon atoms.

Examples of liposoluble homopolymers that may in particular be mentioned are: polyvinyl laurate and lauryl poly(meth)acrylates, these poly(meth)acrylates possibly being cross-linked with the aid of ethylene glycol dimethacrylate or tetraethylene glycol.

In advantageous exemplary embodiments, a composition of the invention includes at least one polyvinyl laurate film-forming polymer.

Examples of liposoluble film-forming polymers that may be used in the invention and that may also be mentioned are polyalkylenes and in particular copolymers of C₂-C₂₀ alkenes such as polybutene, alkylcelluloses with a C₁ to C₈, linear or branched alkyl radical, saturated or unsaturated, such as ethylcellulose or propylcellulose, copolymers of vinylpyrrolidone (VP) and in particular copolymers of vinylpyrrolidone and C₂ to C₄₀ alkene, more preferably C₃ to C₂₀. Examples of VP copolymers that may be used in the invention that may be mentioned are the copolymer of VP/vinyl acetate, VP/ethyl methacrylate, butylated polyvinylpyrolidone (PVP), VP/ethyl methacrylate/methacrylic acid, VP/eicosene, VP/hexadecene, VP/triacontene, VP/styrene and VP/acrylic acid/lauryl methacrylate.

Silicone resins may also be mentioned, generally soluble or swellable in silicone oils, which are cross-linked polyorganosiloxane polymers. The nomenclature of silicone resins is known under the name “MDTQ”, the resin being described as a function of the various monomeric siloxane units it includes, each of the letters “MDTQ” characterizing one type of unit.

Examples of commercially available polymethylsilsesquioxane resins that may be mentioned are those that are sold in particular by the supplier Wacker under the reference Resin MK such as Belsil PMS MK, and by the supplier SHIN-ETSU under the references KR-220L. Siloxysilicate resins that may be mentioned are trimethylsiloxysilicate resins (TMS) such as those sold in particular under the reference SR1000 by the supplier General Electric or under the reference TMS 803 by the supplier Wacker. It is also possible to mention trimethylsiloxysilicate resins in particular sold in a solvent such as cyclomethicone, sold under the name “KF-7312J” by the supplier Shin-Etsu, or “DC 749”, “DC 593” by the supplier Dow Corning.

It is also possible to mention silicone resin copolymers such as those mentioned above with the polydimethylsiloxanes, such as the pressure-sensitive adhesive copolymers sold in particular by the supplier Dow Corning under the reference BIO-PSA and described in the document U.S. Pat. No. 5,162,410, or silicone copolymers derived from the reaction of a silicone resin such as those described above and of a diorganosiloxane such as that described in the document WO 2004/073626.

It is also possible to use silicone polyamides of the polyorganosiloxane type such as those described in documents U.S. Pat. No. 5,874,069, U.S. Pat. No. 5,919,441, U.S. Pat. No. 6,051,216 and U.S. Pat. No. 5,981,680.

These silicone polymers may belong to the following two families:
polyorganosiloxanes comprising at least two groups capables of establishing hydrogen interactions, these two groups being located in the polymer chain; and/or
polyorganosiloxanes comprising at least two groups capable of establishing hydrogen interactions, these two groups being located on grafts or branches.

Vinyl polymers including motifs derived from carbosiloxane dendrimer, such as, for example, those described in documents WO 2006/058793 and EP 1 862 162, are also suitable for use in the invention.

Vinyl polymers grafted with at least one motif derived from carbosiloxane dendrimer that are particularly suitable for the present invention are the polymers sold under the names TIB 4-100, TIB 4-101, TIB 4-120, TIB 4-130, TIB 4-200, FA 4002 ID (TIB 4-202), TIB 4-220, and FA 4001 CM (TIB 4-230) by the supplier Dow Corning.

It is also possible to mention the mixture of decamethylcyclopentasiloxane (40% to 50%) and of polypropylsilanetriol (40% to 50%), sold under the reference DOW CORNING 670 FLUID by the supplier DOW CORNING.

Acrylic copolymers comprising motifs derived from carbosiloxane dendrimer, and in particular copolymers of butyl acrylate comprising dendritic silicone side chains, especially that from the supplier the supplier Dow Corning under the reference FA 4002 ID, may more particularly be used in the invention.

In other exemplary embodiments of the invention, the film-forming polymer is a linear film-forming ethylenic block polymer that preferably comprises at least one first block and at least one second block having different glass transition temperatures (Tg), said first and second blocks being connected together via an intermediate block comprising at least one constituent monomer of the first block and at least one constituent monomer of the second block.

Advantageously, the first and second blocks and the block polymer are incompatibles with each other.

Such polymers are described, for example, in documents EP 1 411 069 or WO 04/028488.

The film-forming polymer may also be present in a composition of the invention in the form of particles in dispersion in an aqueous phase or in a non-aqueous solvent phase, generally known as a latex or pseudolatex. The techniques for preparing such dispersions are well known to the skilled person.

Examples of aqueous dispersions of film-forming polymer that may be used are the acrylic dispersions sold under the names Neocryl XK-90®, Neocryl A-1070®, Neocryl A-1090®, Neocryl BT-62®, Neocryl A-1079® and Neocryl A-523® by the supplier AVECIA-NEORESINS, Dow Latex 432® by the supplier DOW CHEMICAL, Daitosol 5000 AD® or Daitosol 5000 SJ® by the supplier DAITO KASEY KOGYO; Syntran 5760® by the supplier Interpolymer, Allianz OPT by the supplier ROHM & HAAS, aqueous dispersions of acrylic or styrene/acrylic polymers sold under the name JONCRYL® by the supplier JOHNSON POLYMER or aqueous dispersions of polyurethane sold under the names Neorez R-981® and Neorez R-974® by the supplier AVECIA-NEORESINS, Avalure UR-405®, Avalure UR-410®, Avalure UR-425®, Avalure UR-450®, Sancure 875®, Sancure 861®, Sancure 878® and Sancure 2060® by the supplier GOODRICH, Impranil 85® by the supplier BAYER, Aquamere H-1511® by the supplier HYDROMER; sulfopolyesters sold under the name Eastman AQ® by the supplier Eastman Chemical Products, and vinyl dispersions such as Mexomere PAM® from the supplier CHIMEX, and mixtures thereof.

Examples of non-aqueous dispersions of film-forming polymers that may be mentioned are acrylic dispersions in isododecane such as Mexomere PAP® from the supplier CHIMEX, dispersions of particles of a preferably acrylic ethylenic graft polymer, in a liquid fatty phase, the ethylenic polymer advantageously being dispersed in the absence of additional stabilizer on the particle surface in particular such as that described in the document WO 04/055081.

In particularly preferred exemplary embodiments of the invention, the film-forming polymers used in the compositions of the invention are selected from silicone resins, vinyl polymers, in particular of the acrylic type, and acrylic copolymers comprising motifs derived from carbosiloxane dendrimer, and mixtures thereof.

A composition of the invention may also further include a plasticizing agent favoring the formation of a film with the film-forming polymer. Said plasticizing agent may be selected from any compound known to the skilled person to be capable of fulfilling the desired function.

Non-Pulverulent Thickening Agents

The composition of the invention may include one or more non-pulverulent thickening agents or gelling agents.

A thickening agent or gelling agent suitable for the invention may be hydrophilic, i.e. soluble or dispersible in water.

Particular hydrophilic gelling agents that may be mentioned are hydrosoluble or hydrodispersible polymeric thickening agents. They may in particular be selected from: modified or unmodified carboxyvinyl polymers, such as the compositions sold in particular under the names Carbopol (CTFA name: carbomer) by the supplier Goodrich; polyacrylates and polymethacrylates such as the compositions sold under the names Lubrajel and Norgel by the supplier GUARDIAN or under the name Hispagel by the supplier HISPANO CHIMICA; polyacrylamides; polymers and copolymers of 2-acrylamido 2-methylpropane sulfonic acid, optionally cross-linked and/or neutralized, such as poly(2-acrylamido 2-methylpropane sulfonic acid) sold in particular by the supplier CLARIANT under the name “Hostacerin AMPS” (CTFA name: ammonium polyacryldimethyltauramide); cross-linked anionic copolymers of acrylamide and AMPS, in the form of a W/O emulsion, such as those sold in particular under the name SEPIGEL 305 (C.T.F.A. name: Polyacrylamide/C₁₃ to C₁₄ Isoparaffin/Laureth-7) and under the name SIMULGEL 600 (C.T.F.A. name: Acrylamide/Sodium acryloyldimethyltaurate copolymer/Isohexadecane/Polysorbate 80) by the supplier SEPPIC; polysaccharide biopolymers such as xanthan gum, guar gum, carouba gum, acacia gum, scleroglucans, chitin and chitosan derivatives, carrageenans, gellans, alginates, celluloses such as microcrystalline cellulose, carboxymethylcellulose, hydroxymethylcellullose and hydroxypropylcellulose; and mixtures thereof.

A thickening agent or gelling agent that is suitable for the invention may be lipophilic. It may be mineral or organic.

Examples of lipophilic polymeric organic gelling agents are partially or completely cross-linked elastomeric organopolysiloxanes with a three-dimensional structure, such as those sold in particular under the names KSG6®, KSG16® and KSG18® by the supplier SHIN-ETSU, Trefil E-505C® and Trefil E-506C® by the supplier DOW-CORNING, Gransil SR-CYC®, SR DMF10®, SR-DC556®, SR 5CYC Gel®, SR DMF 10 Gel® and SR DC 556 Gel® by the supplier GRANT INDUSTRIES, SF 1204® and JK 113® by the supplier GENERAL ELECTRIC; ethylcellulose such as that sold under the name Ethocel® by the supplier DOW CHEMICAL; polyamide type polycondensates resulting from condensation between a dicarboxylic acid containing at least 32 carbon atoms and an alkylene diamine, in particular ethylene diamine, in which the polymer includes at least one terminal carboxylic acid group esterified or amidified with at least one monoalcohol or a monoamine containing 12 to 30 linear, saturated carbon atoms, in particular ethylene diamine and stearyl dilinoleate copolymers such as that sold in particular under the name Uniclear 100 VG® by the supplier ARIZONA CHEMICAL; galactommannans comprising one to six, and in particular two to four hydroxyl groups per ose, substituted with a saturated or unsaturated alkyl chain, such as guar gum alkylated with C₁ to C₆ alkyl chains, and in particular C₁ to C₃, and mixtures thereof.

Suitable lipophilic thickening agents for the invention that may also be mentioned are copolymers of the polystyrene and polyalkylene type, and more particularly block copolymers of the “diblock”, “triblock” or “radial” type, of the polystyrene and polyisoprene or polystyrene and polybutadiene type such as those sold in particular under the name Luvitol HSB® by the supplier BASF, of the polystyrene and copoly(ethylene-propylene) type such as those sold in particular under the name Kraton® by the supplier KRATON POLYMERS or of the polystyrene and copoly(ethylene-butylene) type, mixtures of triblock and radial (star) copolymers in isododecane such as those sold in particular by the supplier PENRECO under the name Versagel® such as, for example the mixture of butylene and ethylene and styrene triblock copolymer and ethylene and propylene and styrene star copolymer in isododecane (Versagel M 5960).

It is also possible to mention C₃₀-C₄₅ alkyldimethylsilyl polypropylsilsesquioxane silicone resin sold under the reference DOW CORNING SW-8005 C30 RESIN WAX by the supplier DOW CORNING.

Of the lipophilic gelling agents that may be used in a cosmetic composition of the invention, it is also possible to mention esters of dextrin and of fatty acid, such as dextrin palmitates, in those sold under the names Rheopearl TL® or Rheopearl KL® by the supplier CHIBA FLOUR, hydrogenated vegetable oils such as hydrogenated castor oil, fatty alcohols, in particular C₈ to C₂₆, and more particularly C₁₂ to C₂₂, such as, for example, mysrityl alcohol, cetyl alcohol, stearyl alcohol or behenyl alcohol.

In particularly preferred exemplary embodiments, a composition of the invention may comprise at least one lipophilic thickening agent, in particular selected from polystyrene and polyalkylene type copolymers, and more particularly polystyrene and copoly(ethylene-propylene) type copolymers, in particular those sold under the name Kraton® by the supplier KRATON POLYMERS.

These thickening agents may be thixotropic. Such agents, also termed “rheothickeners”, induce an increase in the viscosity of the composition under the effect of increasing shear. These agents may advantageously be used for the purposes of improving the stability of the composition, as well as to improve the appearance of the cosmetic composition, in particular a foundation, on application.

In exemplary embodiments, a composition of the invention may include thickening agents in a quantity of active substance in the range 0.1% to 40% by weight, in particular from 0.2% to 20% by weight, and more particularly from 0.3% to 15% by weight, relative to the total weight of the composition.

Physiologically Acceptable Medium

The compositions of the invention may include a physiologically acceptable medium, i.e. a non-toxic medium that is suitable for being applied to the keratinous material of human beings, and of pleasant appearance, odor and feel.

The physiologically acceptable medium is generally adapted to the manner in which the composition is to be packaged.

A composition of the invention may be in the form of a fluid, for example a paste or liquid, a gel or a cream.

It may be an oil-in-water, a water-in-oil or a multiple emulsion, an aqueous or an oily gel.

These compositions are prepared using the usual methods.

Aqueous Phase

A composition of the invention may comprise an aqueous phase.

The aqueous phase comprises water and/or at least one hydrosoluble solvent.

The term “hydrosoluble solvent” as used in the present invention means a compound that is liquid at ambient temperature and miscible with water (miscibility in water of more than 50% by weight at 25° C. and at atmospheric pressure).

Further, the hydrosoluble solvents that can be used in the compositions of the invention may be volatile.

Examples of hydrosoluble solvents that may be used in the compositions of the invention that may be mentioned are lower monoalcohols containing 1 to 5 carbon atoms such as ethanol and isopropanol, glycols containing 2 to 8 carbon atoms such as ethylene glycol, propylene glycol, 1,3-butylene glycol or dipropylene glycol, C₃-C₄ ketones and C₂-C₄ aldehydes.

The aqueous phase (water and possibly water-miscible solvent) may be present in the composition of the present application in a quantity in the range 1% to 80% by weight relative to the total weight of the composition, preferably in the range 10% to 60% by weight, and more preferably in the range 20% to 50% by weight.

In other exemplary embodiments, a composition of the invention may be anhydrous, i.e. contain less than 2% by weight of water, or even less than 0.5% of water, the water not being added during preparation of the composition, but corresponding to the residual water supplied by the mixed ingredients.

Fatty Phase

The composition of the invention may comprise at least one liquid and/or solid fatty phase.

The fatty phase is generally present in a composition of the invention in a quantity in the range 1% to 50% by weight relative to the total weight of the composition, preferably in the range 5% to 40% by weight, and more preferably in the range 10% to 30% by weight.

The fatty phase of a composition of the invention may in particular comprise one or more oils or organic solvents.

The term “oil or organic solvent”, in the context of the applicator member a non-aqueous body that is liquid at ambient temperature (25° C.) and atmospheric pressure (760 mmHg [millimeter of mercury]).

The oil may be selected from volatile oils and/or non-volatile oils, and mixtures thereof.

The oil or oils may be present in a quantity in the range 1% to 50% by weight, preferably in the range 4% to 30% by weight relative to the total weight of the composition.

In particular exemplary embodiments, a composition of the invention comprises at least one volatile oil.

The term “volatile oil” in the context of the invention means an oil that evaporates in contact with keratinous material in less than one hour, at ambient temperature and atmospheric pressure. The volatile organic solvent or solvents and the volatile oils of the invention are organic solvents and volatile cosmetic oils, liquid at ambient temperature, having non-zero vapor pressure at ambient temperature and atmospheric pressure, in particular in the range 0.13 Pa to 40,000 Pa [pascal] (10⁻³ mmHg to 300 mmHg), in particular in the range 1.3 Pa to 13,000 Pa (0.01 mmHg to 100 mmHg), and more particularly in the range 1.3 Pa to 1300 Pa (0.01 mmHg to 10 mmHg).

In particular, the volatile oils are selected from oils with a rate of evaporation greater than or equal to 0.002 mg/cm²/min [milligram per square centimeter per minute]. The rate of evaporation is measured as follows:
15 g [gram] of oil or a mixture of oils to be tested is introduced into a crystallizer (diameter: 7 cm [centimeter]) placed on a balance in a temperature-regulated (25° C.) and humidity-regulated (relative humidity 50%) chamber of approximately 0.3 m³ [cubic meter].

The liquid is allowed to evaporate freely without stirring, providing ventilation by a fan (speed of rotation 2700 rpm [revolution per minute] and dimensions 80×80×42 mm [millimeter], for example reference 8550 N from PAPST-MOTOREN; the flow rate corresponds to approximately 50 m³/hour) disposed in a vertical position above the crystallizer containing the solvent, the blades being directed towards the crystallizer and at a distance of 20 cm relative to the bottom of the crystallizer.

At regular intervals, the mass of oil remaining in the crystallizer is measured. The rates of evaporation are expressed in mg of oil evaporated per unit surface area (cm²) and per unit time (minute).

The term “non-volatile oil” means an oil remaining on the keratinous material at ambient temperature and atmospheric pressure for at least a few hours and in particular having a vapor pressure of less than 10⁻³ mmHg (0.13 Pa).

These oils may be hydrocarbon oils, silicone oils, fluorinated oils or mixtures thereof.

The term “hydrocarbon oil” means an oil principally containing hydrogen and carbon atoms and possibly oxygen, nitrogen, sulfur or phosphorus atoms. The volatile hydrocarbon oils may be selected from hydrocarbon oils containing 8 to 16 carbon atoms, and in particular C₈-C₁₆ branched alkanes such as C₈-C₁₆ isoalkanes of petroleum origin (also known as isoparaffins), such as isododecane (also termed 2,2,4,4,6-pentamethylheptane), isodecane, isohexadecane and, for example, oils sold under the trade names Isopars or Permethyls, C₈-C₁₆ branched esters, iso-hexyl neopentanoate, and mixtures thereof. Other volatile hydrocarbon oils such as oil distillates, in particular those sold under the name Shell Solt by the supplier SHELL, may also be used. Preferably, the volatile solvent is selected from volatile hydrocarbon oils containing 8 to 16 carbon atoms and mixtures thereof.

It is also possible to use volatile silicones as the volatile oils such as, for example, linear or cyclic volatile silicone oils, in particular those with a viscosity ≦8 centistokes (8×10⁻⁶ m²/s [square meter per second]), and in particular containing 2 to 7 silicone atoms, these silicone oils optionally including alkyl or alkoxy groups containing 1 to 10 carbon atoms. Examples of volatile silicone oil that may be used in the invention and that may be mentioned are octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane and mixtures thereof.

It is also possible to use fluorinated volatile solvents such as nonafluoromethoxybutane or perfluoromethylcyclopentane.

The composition may also comprise at least one non-volatile oil or non-volatile organic solvent, in particular selected from non-volatile hydrocarbon and/or silicone and/or fluorinated oils.

Non-volatile hydrocarbon oils that may in particular be mentioned are:
hydrocarbon oils of vegetable origin such as triesters of fatty acids and glycerol in which the fatty acids may have chain lengths of C₄ to C₂₄, these possibly being linear or branched, saturated or unsaturated; in particular, these oils are wheatgerm, sunflower, grapeseed, sesame seed, corn, apricot, castor, shea butter, avocado, olive or soy oil, sweet almond oil, palm, rapeseed, cotton, hazelnut, macadamia, jojoba, Luzerne, poppy, Hokaido squash, sesame, pumpkin seed, rapeseed, blackcurrant, evening primrose, millet, barley, quinoa, rye, carthame, bancoulier, passiflora, musk rose; or triglycerides of caprylic/capric acids such as those from the supplier the supplier Stearineries Dubois or those sold under the names Miglyol 810, to 812 and 818 by the supplier Dynamit Nobel;
synthesized ethers containing 10 to 40 carbon atoms;
linear or branched hydrocarbons of mineral or synthetic origin such as Vaseline, polydecenes, hydrogenated polyisobutene such as parleam, squalane, and mixtures thereof;
synthesized esters such as oils with formula R₁COOR₂ in which R₁ represents the residue of a linear or branched fatty acid containing 1 to 40 carbon atoms and R₂ represents a hydrocarbon chain, in particular branched, containing 1 to 40 carbon atoms provided that R₁+R₂ is ≧10, such as, for example Purcellin oil (cetostearyl octanoate), isopropyl myristate, isopropyl palmitate, C₁₂ to C₁₅ alcohol benzoate, hexyl laurate, diisopropyl adipate, isononyl isononanoate, 2-ethylhexyl palmitate, isostearate isostearate, octanoates, decanoates or ricinoleates of alcohols or polyalcohols such as propylene glycol dioctanoate; hydroxylated esters such as isostearyl lactate, di-isostearyl malate; and pentaerythritol esters;
fatty alcohols that are liquid at ambient temperature with a branched and/or unsaturated chain containing 12 to 26 carbon atoms, such as octyl dodecanol, isostearyl alcohol, oleic alcohol, 2-hexyldecanol, 2-butyloctanol or 2-undecylpentadecanol;
higher fatty acids such as oleic acid, linoleic acid, linolenic acid;
carbonates;
acetals;
citrates; and
and mixtures thereof.

The non-volatile silicone oils that may be used in the composition of the invention may be non-volatile polydimethylsiloxanes (PDMS), polydimethylsiloxanes comprising alkyl or alkoxy groups, that may be pendent and/or at the end of the silicone chain, groups each containing 2 to 24 carbon atoms, phenyl silicones such as phenyl trimethicones, phenyl dimethicones, phenyl trimethylsiloxy diphenylsiloxanes, diphenyl dimethicones, diphenyl methyldiphenyl trisiloxanes, or 2-phenylethyl trimethylsiloxysilicates.

Particular fluorinated oils that may be used in the invention are fluorosilicone oils, fluorinated polyethers, or fluorinated silicones such as those described in document EP-A-0 847 752.

Fillers

The composition of the invention may also include at least one filler. These fillers serve in particular to modify the rheology or the texture of the composition.

The fillers may be organic or inorganic, of any shape, be they platelet, spherical or oblong, regardless of crystallographic form (for example lamellar, cubic, hexagonal, orthorhombic, etc). Mention may be made of talc, mica, silica, silica surface treated with a hydrophobic agent, kaolin, polyamide powders (Nylon®) (Orgasol® from the supplier Atochem), poly-β-alanine and polyethylene, tetrafluoroethylene polymer powders (Teflon®), lauroyl-lysine, starch, boron nitride, hollow polymeric microspheres such as those formed from polyvinylidene chloride/acrylonitrile, such as Expancel® (Nobel Industrie), acrylic acid copolymers (Polytrap® from the supplier Dow Corning) and silicone resin microbeads (Tospearls® from the supplier Toshiba, for example), particles of polyorganosiloxane elastomers, precipitated calcium carbonate, magnesium carbonate and bicarbonate, hydroxyapatite, hollow silica microspheres (Silica Beads® from the supplier Maprecos), glass or ceramic microcapsules, metallic soaps derived from organic carboxylic acids containing 8 to 22 carbon atoms, preferably 12 to 18 carbon atoms, for example zinc, magnesium or lithium stearate, zinc laurate, and magnesium myristate.

Examples of lipophilic fillers that may be mentioned are modified clays such as modified magnesium silicate (Bentone gel VS38 from the supplier RHEOX), modified hectorites such as hectorite modified by an ammonium chloride of a C₁₀ to C₂₂ fatty acid, such as hectorite modified by di-stearyl di-methyl ammonium chloride such as, for example, that sold in particular under the name Bentone 38V® by the supplier ELEMENTIS or such as that sold in particular under the name “Bentone 38 CE” by the supplier RHEOX or such as that sold in particular under the name Bentone Gel V5 to 5V by the supplier ELEMENTIS.

It is also possible to use a compound susceptible of swelling with heat, in particular thermoexpansible particles such as non-expanded microspheres of vinylidene chloride/acrylonitrile/methyl methacrylate copolymer or acrylonitrile homopolymer copolymer such as, for example, those sold in particular under the references Expancel® 820 DU 40 and Expancel®007WU by the supplier AKZO NOBEL.

The fillers may represent in the range 0.1% to 25%, in particular in the range 0.5% to 10% by weight relative to the total weight of the composition.

Fibers

The compositions in accordance with the invention may also include at least one fiber.

The term “fiber” is intended to mean an object with length L and diameter D such that L is greater than D, and preferably much greater than D, D being the diameter of the circle in which the section of the fiber is inscribed. In particular, the ratio L/D (or form factor) is selected to be in the range 3.5 to 2500, preferably in the range 5 to 500, and more preferably in the range 5 to 150.

The fibers that may be used in the composition of the invention may be fibers of synthetic or natural, mineral or organic origin. They may be short or long, unitary or organized, for example braided, hollow or solid. They may have any shape, in particular a circular or polygonal section (square, hexagonal or octagonal) depending on the specific application envisaged. In particular, their ends are blunted and/or polished to avoid injury.

In particular, the fibers have a length in the range 1 μm [micrometer] to 10 mm, preferably in the range 0.1 mm to 5 mm and more preferably in the range 0.3 mm to 3 mm. Their section may be comprised in a circle with a diameter in the range 2 nm [nanometer] to 500 μm, preferably in the range 100 nm to 100 μm and more preferably in the range 1 μm to 50 μm. The weight or titer of the fibers is usually given in denier or decitex and represents the weight in grams of 9 km of filament. Preferably, the fibers of the invention have a titer selected to be in the range 0.01 to 10 denier, preferably in the range 0.1 to 2 denier and more preferably 0.3 to 0.7 denier.

The fibers that may be used in the compositions of the invention may be selected from rigid or non-rigid fibers they may be of synthetic or natural, mineral or organic origin.

Furthermore, the fibers may optionally be surface treated, coated or not coated, colored or not colored.

Examples of fibers that may be used in the compositions of the invention that may be mentioned are non-rigid fibers such as polyamide fibers (Nylon®) or rigid fibers such as polyimide-amide fibers, such as those sold under the names KERMEL®, KERMEL TECH® by the supplier RHODIA or poly-(p-phenylene-terephthalamide) (or aramide) sold in particular under the name Kevlar® by the supplier DUPONT DE NEMOURS.

The fibers may be present in a quantity lying in the range 0.01% to 10% by weight, relative to the total weight of the composition, in particular in the range 0.1% to 5% by weight, and more particularly in the range 0.3% to 4% by weight.

Lipophilic Structuring Agent

A composition of the invention may comprise at least one structuring agent for the liquid fatty phase selected from a wax, a paste compound, and mixtures thereof.

The term “wax” is used in the context of the present invention to mean a lipophilic fatty compound that is solid at ambient temperature (25° C.) and at atmospheric pressure (760 mmHg, i.e. 10⁵ Pa), with a reversible solid/liquid change of state, in particular having a melting temperature greater than or equal to 30° C., in particular greater than or equal to 55° C., and possibly up to 250° C., especially up to 230° C., and in particular up to 120° C.

By heating the wax to its melting temperature, it is possible to render it miscible with oils and to form a microscopically homogeneous mixture, but on returning the temperature of the mixture to ambient temperature, the wax re-crystallizes in the oils of the mixture.

The waxes may be present in the composition of the invention in a quantity in the range 0.1% to 30% by weight relative to the fatty phase, preferably in the range 1% to 20%.

The values for the melting point in the invention correspond to the melting peak measured using a differential scanning calorimeter (DSC), for example the calorimeter sold under the name DSC 30 by the supplier METLER, with a temperature ramp-up of 5° C. or 10° C. per minute.

In the context of the invention, the waxes may be those generally used in the cosmetic or dermatological fields. In particular, they may be hydrocarbon, silicone and/or fluorinated waxes, optionally including ester or hydroxyl functions. They may also be of natural or synthetic origin.

Illustrative, non-limiting examples of said waxes that may in particular be mentioned are:
waxes of animal origin such as beeswax, vegetable waxes such as carnauba, candellila, ouricury, Japan wax;
mineral waxes, for example paraffin wax, or microcrystalline waxes or ozokerites,
synthetic waxes including polyethylene waxes, and the waxes obtained by Fisher-Tropsch synthesis;
silicone waxes, in particular substituted linear polysiloxanes; mention may be made, for example, of silicone polyether waxes, alkyl or alkoxy-dimethicones containing 16 to 45 carbon atoms, and alkyl methicones such as C30-C45 alkyl methicone sold under the trade name “AMS C 30” from the supplier DOW CORNING,
hydrogenated oils that are solid at 25° C. such as hydrogenated castor oil, hydrogenated jojoba oil, hydrogenated palm oil, hydrogenated tallow, hydrogenated coconut oil and fatty esters that are solid at 25° C. such as C20-C40 alkyl stearate sold under the trade name “KESTER WAX K82H” by the supplier KOSTER KEUNEN, and/or
mixtures thereof.

In exemplary embodiments, the wax present in the composition of the invention may be completely or partially in the powder form, in particular micronized, to facilitate its use in the preparation of the cosmetic composition.

Examples of waxes that may be used in the powder form that may be mentioned are carnauba wax microbeads sold under the name Microcare 350® by the supplier Micro Powders and paraffin wax microbeads sold under the name Microease 114S® by the supplier Micro Powders. Such additional micronized waxes may in particular improve properties during application of the composition to the skin.

The composition of the invention may comprise substituted polysiloxanes, preferably with a low melting point, for example linear substituted polysiloxanes essentially constituted (apart from the terminal groups) by motifs with formulae II and III, in respective molar proportions of m and n:

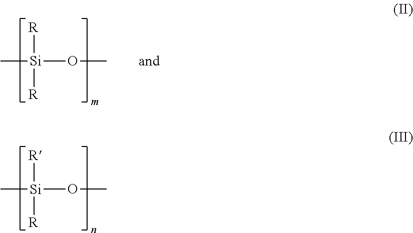

in which:
each substituent R is as defined above;
each R′ independently represents an optionally unsaturated alkyl (linear or branched), containing 6 to 30 carbon atoms, or a —X—R″ group, each X independently representing:
   —O—;
   —(CH₂)ₐ—O—CO—;
   —(CH₂)_{b}—CO—O—;
a and b independently represent numbers that may lie in the range 0 to 6; and each R″ independently represents an optionally unsaturated alkyl group containing 6 to 30 carbon atoms;
m is a number that may lie in the range 0 to 400, and in particular from 0 to 100;
n is a number that may lie in the range 1 to 200, and in particular from 1 to 100; and
the sum (m+n) is less than 400, and in particular less than or equal to 100.

These silicone waxes are known or may be prepared using known methods. Particular examples of commercially available silicone waxes of this type that may be mentioned are those sold under the names Abilwax 9800, 9801 or 9810 (GOLDSCHMIDT), KF910 and KF7002 (SHIN ETSU), or 176-1118-3 and 176-11481 (GENERAL ELECTRIC).

Silicone waxes that may be used may also be selected from compounds with formula (IV):

R₁—Si(CH₃)₂—O—[Si(R)₂—O—]_{z}—Si(CH₃)₂—R₂ (IV)

in which:
R is as defined above;
R₁ represents an alkyl group containing 1 to 30 carbon atoms, an alkoxy group containing 6 to 30 carbon atoms, or a group with formula:

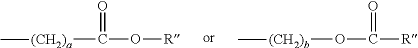

R₂ represents an alkyl group containing 6 to 30 carbon atoms or an alkoxy group containing 6 to 30 carbon atoms or a group with formula:

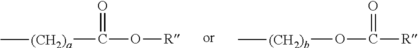

a and b representing a number from 0 to 6;
R″ being an alkyl containing 6 to 30 carbon atoms; and
z being a number that may lie in the range 1 to 100.

Examples of silicone waxes with formula (IV) that are known compositions or may be prepared using known methods that may in particular be mentioned are the following commercially available compositions: Abilwax 2428, 2434 and 2440 (GOLDSCHMIDT), or VP 1622 and VP 1621 (WACKER).

A composition of the invention may further include at least one paste compound.

Said compound may advantageously be selected from lanolin and derivatives thereof; polymeric or non-polymeric silicone compounds; polymeric or non-polymeric fluorinated compounds; vinyl polymers, in particular olefin homopolymers; olefin copolymers; homopolymers and copolymers of hydrogenated dienes; linear or branched oligomers, homo or copolymers of alkyl(meth)acrylates preferably containing a C₈-C₃₀ alkyl group; homo- and copolymeric oligomers of vinyl esters containing C₈-C₃₀ alkyl groups; homo- and copolymeric oligomers of vinyl ethers containing C₈-C₃₀ alkyl groups; liposoluble polyethers resulting from polyetherification between one or more C₂-C₁₀₀ diols, in particular C₂-C₅₀; fatty acid or alcohol esters; and mixtures thereof.

The following esters may in particular be mentioned:
esters of a glycerol oligomer, in particular diglycerol esters, such as polyglyceryl-2 triisostearate, condensates of adipic acid and glycerol, in which a portion of the hydroxyl groups of the glycerols has been reacted with a mixture of fatty acids such as stearic acid, capric acid, stearic and isostearic acid and 12-hydroxystearic acid, especially similar to those sold with reference Softisan 649 by the supplier Sasol or such as bis diglyceryl polyacyladipate-2; arachidyl propionate sold in particular with reference Waxenol 801 by the supplier Alzo; phytosterol esters; triglycerides of fatty acids and derivatives thereof, such as hydrogenated coco-glycerides; non cross-linked polyesters resulting from polycondensation between a dicarboxylic acid or a linear or branched C₄-C₅₀ carboxylic polyacid and a C₂-C₅₀ diol or a polyol; aliphatic ester esters resulting from the esterification of an ester of an aliphatic hydroxycarboxylic acid by an aliphatic carboxylic acid; polyesters resulting from esterification, by a polycarboxylic acid, of an aliphatic hydroxy carboxylic acid ester, said ester including at least two hydroxyl groups, such as the products Risocast DA-H®, and Risocast DA-L®; and mixtures thereof.

The structuring agent or agents may be present in a composition of the invention in a quantity in the range 0.1% to 30% by weight of agents, more preferably from 1% to 20% by weight, relative to the total weight of the composition.

Additional Polymers

The composition of the invention may further contain a silicone elastomer.

This elastomer may be polyglycerolated. By way of example, a cross-linked organopolysiloxane elastomer is used that may be obtained by addition cross-linking of a diorganopolysiloxane containing at least one hydrogen bonded to silicone and polyglycerolated compounds containing groups with an ethylenically unsaturated bond, in particular in the presence of a platinum catalyst.

Examples of polyglycerolated silicone elastomers that may be used are those sold under the names “KSG-710”, “KSG-810”, “KSG-820”, “KSG-830”, “KSG-840” by the supplier Shin Etsu.

The compositions of the invention may further comprise an additional emulsifying silicone elastomer.

This silicone elastomer may optionally have an emulsifying effect.

Examples of silicone elastomers having an emulsifying effect and that may be used are polyoxyalkylene silicone elastomers as described in particular in U.S. Pat. No. 5,236,986, U.S. Pat. No. 5,412,004, U.S. Pat. No. 5,837,793, and U.S. Pat. No. 5,811,487.

Examples of polyoxyalkylene elastomer silicones that may be used are those sold in particular under the names “KSG-21”, “KSG-20”, “KSG-30”, “KSG-31”, “KSG-32”, “KSG-33”, “KSG-210”, “KSG-310”, “KSG-320”, “KSG-330”, “KSG-340”, “X-226146” by the supplier Shin Etsu, “DC9010”, “DC9011” by the supplier Dow Corning.

Examples of non-emulsifying silicone elastomers that may be mentioned are the silicone elastomers described in applications JP-A-61-194009, EP-A-0 242 219, EP-A-0 285 886 and EP-A-0 765 656.

Examples of spherical non-emulsifying elastomers that may be used are those sold under the names “DC 9040”, “DC9041”, “DC 9509”, “DC9505”, “DC 9506” by the supplier Dow Corning.

The spherical non-emulsifying silicone elastomer may also be in the form of powder of a cross-linked organopolysiloxane elastomer coated with silicone resin, in particular silsesquioxane resin as described, for example, in U.S. Pat. No. 5,538,793. Such elastomers are sold under the names “KSP-100”, “KSP-101”, “KSP-102”, “KSP-103”, “KSP-104”, “KSP-105” by the supplier Shin Etsu.

Other cross-linked organopolysiloxane elastomers in the form of spherical powders may be hybrid silicone powders functionalized with fluoroalkyl groups, sold in particular under the name “KSP-200” by the supplier Shin Etsu; or hybrid silicone powders functionalized with phenyl groups, in particular sold under the name “KSP-300” by the supplier Shin Etsu.

Other Ingredients

The compositions of the invention may further include any cosmetic active ingredient such as active ingredients selected from antioxidants, preservatives, fragrances, bactericidal or antiperspirant active ingredients, neutralizers, emollients, moisturizers, oligo-elements, softeners, sequestrating agents, alkalizing or acidifying agents, hydrophilic or lipophilic active ingredients, coalescence agents, surfactants, plasticizers, vitamins, screens, in particular sunscreens, and mixtures thereof.

According to a particular embodiment of the invention, the composition of the invention is different from deodorant and antiperspirant formulations.

In particular, a composition of the invention may advantageously comprise less than 2% by weight of deodorant or antiperspirant actives relative to the total weight of the composition, in particular less than 1% by weight and more preferably is totally free of deodorant or antiperspirant actives.

Said actives that are termed “antiperspirant” or “deodorant” are well known to the skilled person. Antiperspirant or deodorant actives that may be mentioned include active salts such as aluminium or zirconium salts, bacteriostatic agents and bactericidal agents such as quaternary ammonium salts, etc.

According to an other particular embodiment, the composition of the invention is different from a soap composition.

Clearly, the skilled person will be careful to select any complementary compounds and/or their quantity in a manner such that the envisaged addition degrades the advantageous properties of the composition of the invention little or not at all.

The invention is illustrated in the examples below by way of non-limiting illustration of the invention:

Unless otherwise indicated, the values in the examples below are expressed as a % by weight relative to the total weight of the composition.

### EXAMPLE 1

Fluid foundation composition including a large quantity of pigments.

| Ingredients/Commercial references | % by weight |
|---|---|
| Magnesium sulfate, 7 H₂O | 1.5 |
| Hectorite-modified distearyl dimethyl ammonium sold | 1 |
| under the reference BENTONE 38 VCG by | |
| ELEMENTIS | |
| Yellow, red and black oxides of iron and oxides of | 24 |
| titanium respectively coated with aluminum stearoyl | |
| glutamate (97/3) (sold under the references NAI-C33 | |
| by MIYOSHI KASEI) | |
| Mixture of oxyethylenated polymethylcetyl dimethyl | 9 |
| methylsiloxane, polyglyceryl iso-stearate (4 moles), | |
| hexyl laurate (sold under the reference ABIL WE 09 by | |
| EVONIK GOLDSCHMIDT) | |
| Sodium carboxymethyl cellulose (sold under the | 0.5 |
| reference BLANOSE 7M8SF by AQUALON | |
| (HERCULES)) | |
| Expanded micro-spheres of vinylidene chloride/ | 0.6 |
| acrylonitrile/PMMA with isobutane (EXPANCEL 551 | |
| DE 20 D60 from Expancel) | |
| Preservatives | 3 |
| Glycerol | 5 |
| Water | 31.7 |
| Mixture of acetylated ethylene glycol stearate, glyceryl | 0.3 |
| tristearate (sold under the reference UNITWIX by | |
| United Guardian) | |
| Cyclopentadimethylsiloxane (sold under the reference | Qsp 100 |
| XIAMETER PMX-0245 CYCLOSILOXANE by | |
| DOW CORNING) | |

Operating Method:

The pigment paste was prepared using a three cylinder mill by dispersing the pigments in silicone. Next, the mixture was passed through the three cylinder mill three times.

Preparation of the Fatty Phase

The hectorite was dispersed in the remaining cyclopentasiloxane with magnetic stirring for 10 min.

The mixture of oxyethylenated polymethylcetyl dimethyl methylsiloxane, polyglyceryl iso-stearate (4 moles), hexyl laurate and the mixture of acetylated ethylene glycol stearate, glyceryl tri-stearate was prepared and heated in a water bath to 80° C. then cooled to 35° C.

The various phases and the pigment paste were mixed and the mixture was homogenized in a turbine for 15 minutes.

Next, the Expancel was pre-mixed in cyclopentasiloxane for 5 minutes with stirring.

Preparation of the Aqueous Phase

The carboxymethyl cellulose was added to the water, dispersed in the cold then heated to 60° C. to allow homogenization and complete hydration. The preservatives, glycerin and water were mixed with magnetic stirring and heated to 85° C. to dissolve the preservatives and homogenize the mixture.

The various phases were mixed, the homogenized mixture was then cooled to 25° C. to 30° C.

Emulsion

Finally, the aqueous phase was slowly added to the fatty phase. It was allowed to emulsify for 10 min in a bath of cold water.

This composition had a viscosity of 4.7 Pa·s.

Results

Application of this composition with an applicator device provided with a rotary means as described in FIGS. 1 to 3 produced a makeup with very good coverage.

The film applied was thin, uniform and smooth. The makeup was discrete and natural.

Application of this composition with an applicator device as described in FIGS. 1 to 3 was compared with the application with a conventional sponge.

Application was carried out using an applicator device over half of the face, the other half being made up using a conventional sponge.

Said sponge and applicator device were weighted before and after the application over each half of the face so that the same quantity (0.20 g) of composition is applied over each half of the face.

The application sensations (softness, slippery effect), makeup result (uniformity, coverage) and wear property after eight hours were evaluated by the user; symbols “+” to “+++” being attributed according to satisfaction of the user.

In terms of the coverage property, it is appreciated visually according to change in color of the skin, masking of relief imperfections of the skin, etc.

In terms of wear property after eight hours, it is evaluated both according to visual changes in color, glossy effect of the skin and according to the quantity of removed composition during makeup removal: a makeup removing cotton pad is applied over each of the face and the quantity of removed makeup composition on the cotton pad is appreciated. A lot of makeup on the cotton pad means that the makeup composition remains over the skin up to the makeup removal and this indicates good wear properties before makeup removal.

A table 1 is reported below, giving the noted results for the two applications.

TABLE 1

| | | Application with the |
|---|---|---|
| | Application with a | applicator device of the |
| Evaluation | conventional sponge | invention |
| Uniformity of the | + | +++ |
| deposit | | |
| Uniformity at the | ++ | +++ |
| end of the makeup | | |
| Application softness | + | ++ |
| Application | + | +++ |
| slippery effect | | |
| Coverage property | ++ | +++ |
| Wear property | ++ | +++ |

In particular, it is much more difficult to get a makeup with satisfactory uniformity by applying the composition with a conventional sponge in comparison with the application with the applicator device of the invention.

In terms of the makeup results, the makeup resulting from application with the applicator device of the invention presents a better uniformity.

### EXAMPLE 2

Fluid foundation composition comprising a large quantity of pigments+non-pulverulent thickening agent+film-forming polymer.

| Ingredients/Commercial references | % by weight |
|---|---|
| Magnesium sulfate, 7 H₂O | 0.70 |
| Smectite: modified magnesium silicate in isododecane | 8.00 |
| (sold under the reference BENTONE GEL ISD V by | |
| ELEMENTIS) | |
| Yellow, red and black iron oxides and titanium oxide | 14 |
| respectively coated with aluminum stearoyl glutamate | |
| (97/3) (sold under the references NAI-C33 by MIYOSHI | |
| KASEI) | |
| Preservatives | 0.24 |
| Butyl acrylate copolymer containing dendritic silicone | 12.50 |
| side chains: Tri (trimethylsiloxy) siloxy ethyl dimethyl | |
| siloxy silyl propyl-methacrylate in isododecane: 40/60 | |
| (sold under the reference DOW CORNING FA 4002 ID | |
| SILICONE ACRYLATE by DOW CORNING) | |
| Polydimethylsiloxane with alpha-omega oxyethylene/ | 1.80 |
| oxypropylene groups in solution in cyclopentasiloxane | |
| (sold under the reference ABIL EM 97 by EVONIK | |
| GOLDSCHMIDT) | |
| Nylon 12 powder | 1.10 |
| Styrene/ethylene-propylene (37/63) diblock copolymer | 0.90 |
| (sold under the reference KRATON G1701 E by | |
| KRATON POLYMERS) | |
| Mono/diglycerides of isostearic acid esterified with | 0.60 |
| succinic acid (sold under the reference IMWITOR 780 K | |
| by SASOL) | |
| Octane-1,2-diol | 0.29 |
| Iso-dodecane | 9.45 |
| Glycerol | 3.00 |
| Water, deionized | 25.24 |
| Iso-hexadecane | 4.99 |
| Cyclopentadimethylsiloxane (sold under the reference | Qsp 100 |
| XIAMETER PMX-0245 CYCLOSILOXANE by DOW | |
| CORNING) | |

Operating Method:

The isohexadecane and then the Kraton were introduced into a pan placed in an oil bath heated to 90° C. The mixture was allowed to swell for approximately 2 hours.

The preservatives were dissolved with the polydimethylsiloxane with alpha-omega oxyethylene/oxypropylene groups in solution in cyclopentasiloxane and the mono/diglycerides of isostearic acid esterified with succinic acid in the water bath. The butyl acrylate silicone polymer and the smectite were introduced bit by bit into a small turbine, and allowed to disperse for approximately 10 to 15 minutes.

All of the ingredients of the aqueous phase were brought to the boil and allowed to return to ambient temperature.

The pigments were milled with the cyclopentasiloxane with the three cylinder mill (3 passes), introduced into the fatty phase and dispersed for 30 minutes with the aid of a band mill. The fillers were then added and dispersed again over 5 to 10 minutes.

Finally, the aqueous phase and the fatty phase were mixed at ambient temperature for 10 minutes.

This composition had a viscosity of 0.7 Pa·s. Further, it had the disadvantage of drying very rapidly on application to keratinous material, and proved difficult to apply uniformly. The user in effect had a limited time available for making the thickness of the deposit uniform once the composition had been applied.

Results

Application of this composition with an applicator device provided with a rotary means as described in FIGS. 1 to 3 enables a makeup to be obtained with very good coverage.

The film applied was thin, uniform and smooth. The makeup was discrete and natural.

### EXAMPLE 3

Fluid foundation composition comprising a large quantity of pigments+non-pulverulent thickening agent+film-forming polymer.

| Ingredients/Commercial references | % by weight |
|---|---|
| Sodium chloride | 1 |
| Hectorite-modified distearyl dimethyl ammonium (sold | 0.6 |
| under the reference BENTONE 38 VCG by ELEMENTIS) | |
| Yellow, red and black iron oxides and titanium oxide | 10 |
| respectively coated with aluminum stearoyl glutamate | |
| (97/3) (sold under the references NAI-C33 by MIYOSHI | |
| KASEI) | |
| Preservatives | 1.1 |
| C₃₀-C₄₅ alkyldimethylsilyl polypropylsilsesquioxane (sold | 4 |
| under the reference DOW CORNING SW-8005 C30 | |
| RESIN WAX by DOW CORNING) | |
| Decamethylcyclopentasiloxane (40%-50%) and | 12 |
| polypropylsilanetriol resin (40%-50%) (sold under the | |
| reference DOW CORNING 670 FLUID by DOW | |
| CORNING) | |
| Polyglycerin cross-linked with silicone chains (sold under | 6 |
| the reference KSG 710 by Shin Etsu) | |
| Expanded micro-spheres of vinylidene chloride/ | 0.6 |
| acrylonitrile/PMMA with iso-butane (EXPANCEL 551 | |
| DE 20 D60 from Expancel) | |
| Iso-dodecane | 5 |
| Glycerol | 3 |
| Water, deionized | 31.9 |
| Ethylene diamine tetraacetic acid, disodium salt, 2 H₂O | 0.2 |
| Cyclopentadimethylsiloxane (sold under the reference | Qsp 100 |
| XIAMETER PMX-0245 CYCLOSILOXANE by DOW | |
| CORNING) | |

Operating Method:

The preservatives were dissolved with the other compounds constituting the fatty phase in a water bath.

All of the ingredients of the aqueous phase were brought to the boil and allowed to return to ambient temperature.

The pigments were milled with the cyclopentasiloxane using the three cylinder mill (3 passes), then introduced into the fatty phase and dispersed for 30 minutes with the aid of a band mill. The fillers were then added and dispersed again over 5 to 10 minutes.

Finally, the aqueous phase and the fatty phase were mixed at ambient temperature for 10 minutes.

This composition had a viscosity of 0.8 Pa·s. It also had a tacky texture that could render smoothing of the composition using conventional applicator means (finger, sponge) difficult.

Results

Application of this composition with an applicator device provided with a rotary means as described in FIGS. 1 to 3 was evaluated by a panel of 6 women in the laboratory.

Application was carried out using an applicator device over half of the face, the other half being made up using the fingers.

The application, perceptions and the makeup result for the two faces were evaluated.

The applicator device could take foundation composition more easily. It also had the advantage of not soiling the fingers with foundation.

Further, the composition was easier to apply with the applicator device of the invention.

In terms of the makeup result, the makeup had very good coverage. Uniformity was better when the composition was applied using the applicator device. The applied film was then thin, uniform and smooth. The makeup was discrete and natural.

Finally, the users appreciated the slight massage provided by the light strokes employed on application of said composition with this device.

1. A kit for making up and/or for care of keratinous material, comprising:
a container containing at least one cosmetic composition comprising, in a physiologically acceptable medium, at least 16% by weight of pigments and/or of film-forming polymers and/or of non-pulverulent thickening agents, relative to the total weight of said composition, said composition comprising at least one pigment; and
an applicator device configured to apply the composition, the applicator device comprising an applicator member having an application surface configured to turn about at least one axis or center of rotation in response to being moved into engagement with keratinous material.

2. The kit according to claim 1, wherein the composition is a fluid.

3. The kit according to claim 1, wherein the composition includes at least 16% by weight of pigments relative to the total weight of said composition, and wherein said composition at 25° C. and at a shear rate of 1 s⁻¹, has a viscosity of more than 3 Pa·s.

4. The kit according to claim, wherein the composition includes at least 20% by weight of pigments, relative to its total weight.

5. The kit according to claim 1, wherein the composition includes at least 10% by weight of film-forming polymers and/or of non-pulverulent thickening agents, relative to its total weight.

6. The kit according to claim 1, wherein the pigments are selected from yellow, red and black iron oxides and titanium dioxide.

7. The kit according to claim 1, wherein the film-forming polymers are selected from silicone resins, vinyl polymers, and acrylic copolymers comprising motifs derived from carbosiloxane dendrimer.

8. The kit according to claim 1, wherein the non-pulverulent thickening agents are selected from lipophilic thickening agents.

9. The kit according to claim 1, wherein the composition is chosen from a blusher, an eye shadow, a foundation, a concealer, a lipstick, a body makeup composition, a care product for the face, a care product for the body and a sunscreen.

10. The kit according to claim 1, wherein the composition is a foundation.

11. The kit according to claim 1, wherein the applicator member is a roller.

12. The kit according to claim 11, wherein the roller has an application surface chosen from an elastomeric material, a foam with open or closed cells, a flocked membrane, a sintered material, a woven material and a nonwoven material.

13. A method of making-up and/or caring for a keratinous material, the method comprising:
loading an applicator member with a quantity of a solid composition with the applicator member having an application surface configured to turn about an axis or a center of rotation in response to being moved into engagement with keratinous material; and
applying the composition to keratinous material with said applicator member, said composition comprising, in a physiologically acceptable medium, at least 16% by weight of pigments and/or of polymers and/or of non-pulverulent thickening agents, relative to the total weight of said composition, and said composition comprising at least one pigment.

14. The method according to claim 13, wherein said composition is a fluid.

15. The kit according to claim 1, wherein the composition comprises at least 18% by weight of pigments and/or of film-forming polymers and/or of non-pulverulent thickening agents, relative to the total weight of said composition.

16. The kit according to claim 1, wherein the composition comprises at least 20% by weight of pigments and/or of film-forming polymers and/or of non-pulverulent thickening agents, relative to the total weight of said composition.

17. The kit according to claim 3, wherein the composition has a viscosity ranging from 3 Pa·s to 6 Pa·s.

18. The kit according to claim 4, wherein the composition includes a range of 22% to 65% by weight of pigments relative to its total weight.

19. The kit according to claim 4, wherein the composition includes a range of 24% to 45% by weight of pigments relative to its total weight.

20. The kit according to claim 5, wherein the composition includes a range of 10% to 30% by weight of film-forming polymers and/or of non-pulverulent thickening agents, relative to its total weight.

21. The kit according to claim 5, wherein the composition includes a range of 12% to 20% by weight of film-forming polymers and/or of non-pulverulent thickening agents, relative to its total weight.

22. The kit according to claim 6, wherein the pigments are coated with at least one hydrophobic agent chosen from aluminum acyl glutamate and aluminum stearoyl glutamate.

23. The kit according to claim 7, wherein the vinyl polymers comprise acrylic vinyl polymers.

24. The kit according to claim 8, wherein the non-pulverulent thickening agents are selected from polystyrene/polyalkylene and polystyrene/copoly(ethylene-propylene) copolymers.

25. The method according to claim 13, wherein the composition includes at least 16% by weight of pigments relative to the total weight of said composition, and wherein said composition at 25° C. and at a shear rate of 1 s⁻¹, has a viscosity of more than 3 Pa·s.

26. The method according to claim 25, wherein the composition has a viscosity ranging from 3 Pa·s to 6 Pa·s.

27. The method according to claim 13, wherein the composition comprises at least 18% by weight of pigments and/or of polymers and/or of non-pulverulent thickening agents, relative to the total weight of said composition.

28. The method according to claim 13, wherein the composition comprises at least 20% by weight of pigments and/or of polymers and/or of non-pulverulent thickening agents, relative to the total weight of said composition.

29. The method according to claim 13, wherein the composition includes at least 20% by weight, of pigments, relative to its total weight.

30. The method according to claim 13, wherein the composition includes at least 10% by weight of polymers and/or of non-pulverulent thickening agents, relative to its total weight.

31. The method according to claim 13, wherein the pigments are selected from yellow, red and black iron oxides and titanium dioxide.

32. The method according to claim 31, wherein the pigments are coated with at least one hydrophobic agent chosen from aluminum acyl glutamate and aluminum stearoyl glutamate.

33. The method according to claim 13, wherein the polymers are selected from silicone resins, vinyl polymers, and acrylic copolymers comprising motifs derived from carbosiloxane dendrimer.

34. The method according to claim 13, wherein the non-pulverulent thickening agents are selected from lipophilic thickening agents.

35. The method according to claim 13, wherein the composition is chosen from a blusher, an eye shadow, a foundation, a concealer, a lipstick, a body makeup composition, a care product for the face, a care product for the body and a sunscreen.

36. The method according to claim 13, wherein the composition is a foundation.
